(19) United States
(12) Patent Application Publication
(10) Pub. No.: US 2005/0239184 A1
(43) Pub. Date: Oct. 27, 2005

(54) Electric driven protein immobilizing module and method

(76) Inventors: Reiko Ohara, Hsinchu (TW); Chiun-Jye Yuan, Hsinchu City (TW); Wei-Jen Ho, Hsinchu County (TW)

Correspondence Address: BIRCH STEWART KOLASCH & BIRCH, PO BOX 747, FALLS CHURCH, VA, 22040-0747, US

(21) Appl. No.: 10/829,988

(22) Filed: Apr. 23, 2004

(51) Int. Cl. C12N 11/06

(52) U.S. Cl. 435/174; 204/450

(57) ABSTRACT

An electric driven protein immobilizing module and method aims at immobilizing proteins rapidly and steadily on the surface of a selected support. The invention employs the characteristics of proteins/enzymes forming a slightly negative charge in a buffer solution. An external electric field is set up to drive the proteins/enzymes to be adsorbed onto the support. The invention improves upon conventional absorption or bonding methods that fix the protein in a non-directional approach which results in masking the protein active site and subsequently loss the protein activity. Thus activity of the protein/enzyme improved, while the time-consuming problem and enzymatic activity loss problem of incubation and vacuum absorption method may be avoided.

### FIELD OF THE INVENTION

The present invention relates to a protein immobilizing module and method, and particularly to an apparatus and method that employ an external electric field to move the protein/enzyme and shorten the diffusion time.

### BACKGROUND OF THE INVENTION

Proteins are mainly composed of amino and carboxylic acid functional group. Hence immobilizing the protein generally is accomplished by forming a bond between the amino (—NH₂) and carboxylic (—COOH) group on a support. In general, the methods for immobilizing proteins can be divided into three types.

The first type is carrier-binding which immobilizes the protein on an insoluble support (i.e. solid type). Carrier-binding methods further can be grouped in three categories:

a. Physical adsorption: which adsorbs the protein through physical characteristics such as van der waals interaction or hydrogen bonding. It has the advantages of low cost and also the bond can be formed easily. However it has a drawback of weak adsorption binding force. The protein is prone to peel off from the support due to external factors such as changes of temperature, pH value, and ionic concentration in the solution.

b. Ionic bonding: the protein bonds on the support with an ionic bonding. It has the advantages of simple operation and smaller effect on the conformational change of the protein. However, the result is sensitive to the changes of pH value, ionic concentration and temperature. Nevertheless, it provides a stronger bonding force/interaction than the physical adsorption.

c. Covalent bonding: Some of the functional groups (such as amino and carboxylic acid group) do not play any role in the activity of the protein. Therefore they may be used to form a covalent bond with the functional groups which are already existed on the surface of the support. Such a bonding is stronger and can immobilize the protein without desorbing from the support when subject to external factors. However, the support cannot be regenerated and reused.

The second type is cross-linking. The protein is cross-linked with a bi- or multifunctional groups to achieve the immobilizing effect. However, the protein loses its enzymatic activity easily.

The third type is entrapment which entraps protein in closed or porous polymers. This type can be grouped in two categories as follows:

a. Lattice-type which entraps the protein in a polymeric gel lattice or a crosslinked polymeric network lattice.

b. Micro-capsule-type which envelops the protein in small granules or capsules.

All of the techniques for immobilizing protein set forth above have two main common problems. First, the active sites of the protein/enzyme is randomly (non-orient) adsorbed or covalent-bonded on the selected support. This surface would promote a high steric hindrance. Secondly, in the general immobilizing processes, incubation is the most widely adopted method. However, this method needs to incubate the protein for several hours so that the protein could be diffused and distributed evenly to the support in order to achieve the optimal immobilizing efficiency. To some supports (such as filter paper or semi-permeable membrane), the incubation approach could lead to planar (lateral) diffusion on the support and result in non-uniform (uneven) distribution of the protein/enzyme on the support. Another approach is vacuum suction which can save time and is more versatile. However, it is suitable only to the adsorption method or porous supports. Moreover, such approach could result in leakage of the protein/enzyme through the pores of the support under forceful suction. The disadvantage of said conventional methods for protein immobilization is the lengthy time for the protein/enzyme to bind to the support. Most importantly, this can influence the activity of the enzyme. Furthermore, the protein/enzyme 5 (referring to FIG. 1) does not bond with the support 3 in a certain direction (orientation). As a result, the activated portion 50 of the protein/enzyme 5 could promote a steric hindrance and diminishes activity.

### SUMMARY OF THE INVENTION

The primary objective of this specific invention is to provide a protein immobilizing module driven by an electric field. According to the invention, in a solution environment, an electric field may be used to control the orientation of protein/enzyme and accelerate the adsorption of the protein/enzyme to a selected support. This can resolve the problem of diminishing enzymatic activity which caused by masking the active site of the protein/enzyme, and also shortening the protein/enzyme diffusion time.

The present invention employs a module which has an upper tank and a lower tank. The upper tank has an opening and a plurality of sample wells on the bottom. The lower tank is located under the upper tank and has a plurality of apertures corresponding to the sample wells. A selected support is located on the contact surfaces of the upper and lower tanks and is fastened on the periphery by fasteners. The upper and lower modules have respectively an electrode. In the module, a buffer solution is added to form a solution environment.

Protein/enzyme is dissolved in a solution, and then dripped into the sample wells by micropipettes, and an electric current is applied. The protein/enzyme has charges in the solution that may be driven by an external electric field to move in a certain direction towards the selected support. The selected support is anchored on the module. The surface of the chosen support charges which is opposite to the charges of the protein/enzyme. Thus the protein/enzyme may be attracted to the support surface in a direction by electric field.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of a conventional method for immobilizing protein/enzyme.

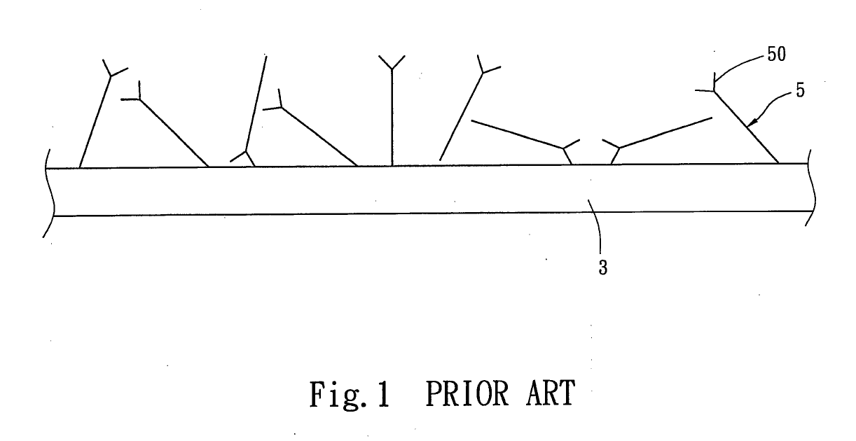

FIG. 2 is a perspective view of a structure embodiment of the present invention.

FIG. 3 is a sectional view of an embodiment of the present invention.

FIG. 4 is a schematic view of an embodiment of the present invention for immobilizing protein/enzyme.

FIG. 5 is a chart showing enzyme light absorption comparisons in different electric fields and a conventional vacuum absorption immobilizing method.

### DETAILED DESCRIPTION OF THE PREFERRED EMBODIMENTS

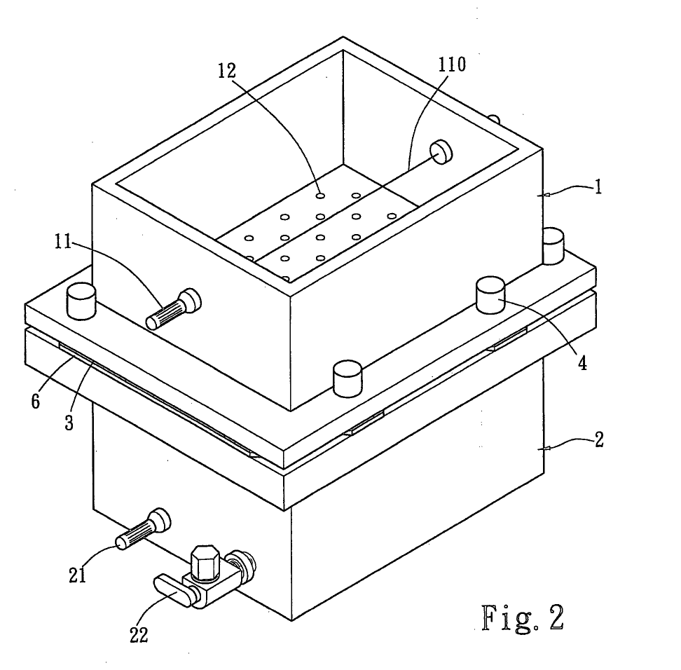

Please refer to FIG. 2 for the structure of the module according to the invention. It includes an upper tank 1 and a lower tank 2. The upper tank 1 can open on the upper side and the lower side. The lower tank 2 is closed and has only one end communicating with the upper tank 1. The upper tank 1 has a first electrode 11 which is a cathode. The first electrode 11 is connected to a platinum wire 110 which is mainly to generate an electric field. The platinum wire 110 may also be replaced by a conductive metal plate. The lower tank 2 has a second electrode 21 which is an anode. The two modules are interposed by a silicon rubber pad 6 to prevent leaking. A selected support 3 is located on the surface of the silicon rubber pad 6 and is a porous membrane, porous powders or porous granules. In the embodiment, the support 3 is a porous membrane which may be made from cellulose nitrate, nylon, Polyvinylidene fluoride (PVDF), cellulose, or combinations thereof. The porous powders may consist of ceramics, alumina, silica, graphite, or combinations thereof. The porous granules may be ceramic, glass, alumina, silica, graphite, or combinations thereof.

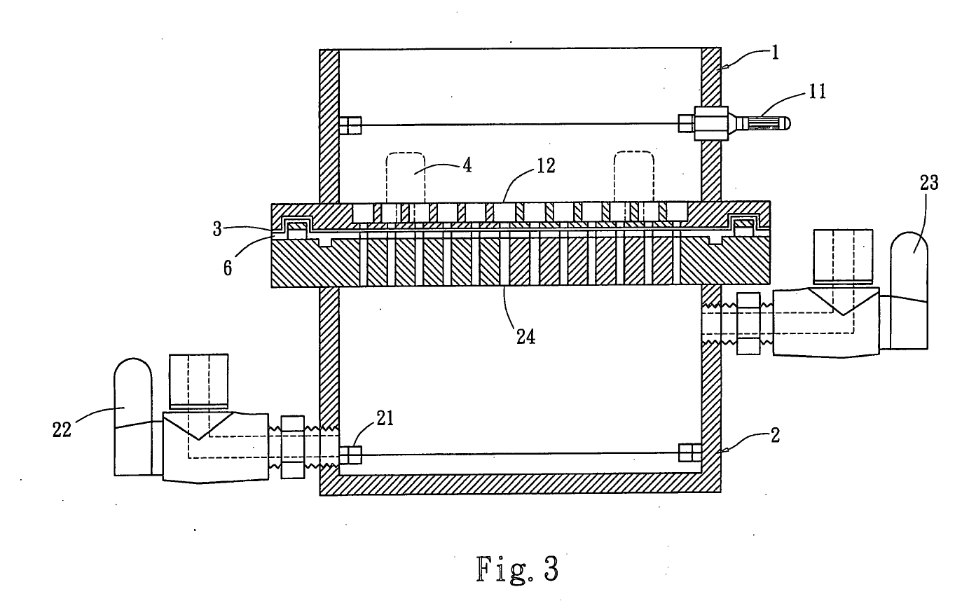

The upper tank 1 has a surface in contact with the support 3 that has a plurality of sample wells 12 formed thereon. The lower tank 2 also has a surface in contact with the support 3 that has a plurality of apertures 24 formed thereon corresponding to the sample wells 12. The silicon rubber pad 6 also has a plurality of ports corresponding to the sample wells 12. The upper and lower tanks 1 and 2 are fastened by a plurality of fasteners 4. The sample wells 12 on the upper tank 1 and the ports on the silicon pad 6, and the apertures 24 on the lower tank 2 are aligned and communicate with one another (also referring to FIG. 3).

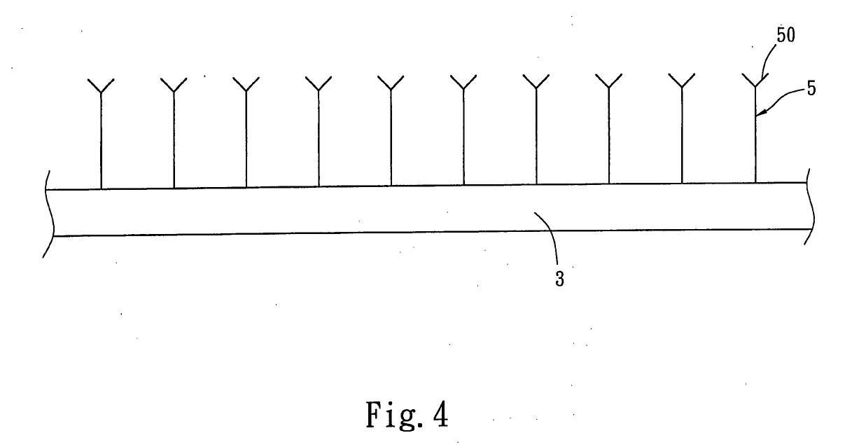

Referring to FIG. 3, the selected support 3 has electric charges which are opposite to the charges of the protein/enzyme. With the selected support 3 located on the silicon rubber pad 6, buffer solution may flow in through a liquid intake switch 23 to form a solution environment with the liquid level submerging the first electrode 11. The buffer solution may be selected from Phosphate buffer, Tris (hydroxymethyl) aminomethane buffer (Tris buffer) and the N-2-hydroxyethylpiperazine-N′-2-ethane sulfonic acid buffer (HEPES buffer). The pH value of the buffer solution is between 7 and 11. The protein/enzyme to be immobilized has an isoelectric point (pI) less than 7. Hence the protein/enzyme in the buffer solution forms a slightly negative charge. This is helpful to the process of immobilizing the protein in a certain direction by electrophoresis effect. The voltage of electric field is controlled in the range of 80 to 350 volts. The resulting electric current density is between 38 mA to 113 mA. After having been electrified for fifteen minutes, the buffer solution is discharged through the outlet 22. Referring to FIG. 4, the invention enables the orientation of the protein/enzyme 5 to be adsorbed on a support in a certain direction. Its activated portions 50 do not interfere with each other or subject to steric hindrance, thus activity does not diminish.

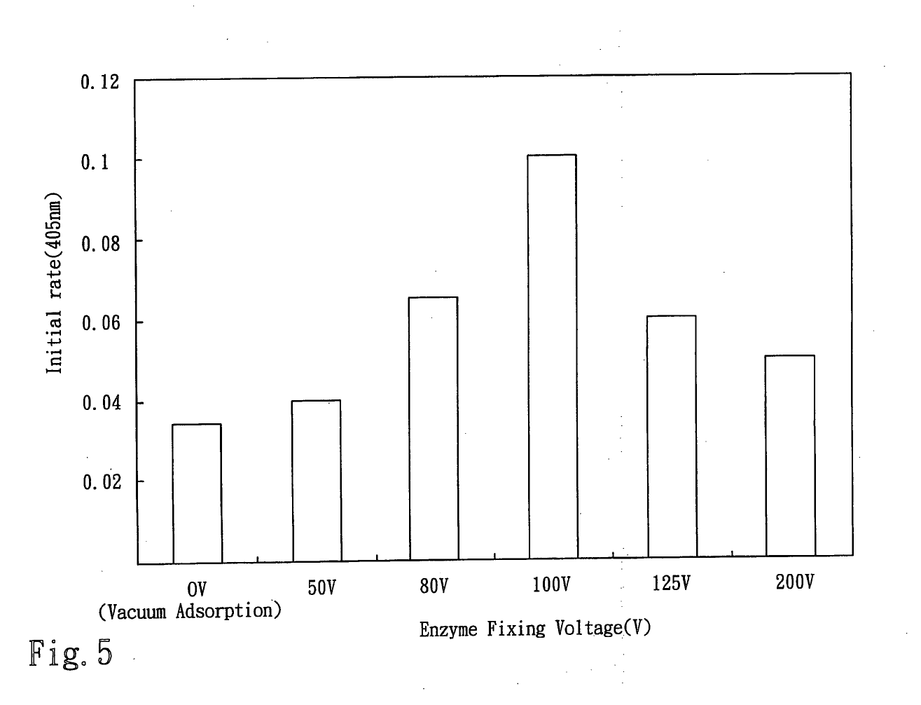

Refer to FIG. 5 for comparisons between the operating results of the invention and the general vacuum absorption immobilizing approach. An enzyme of Acetylcholinesterase (AchE) with pH of 7.2 is selected, the buffer solution is 50 mM of Tris (hydroxymethyl) aminomethane-hydrochloride buffer (Tris-HCl buffer) including 0.2 mM of Acetylcholine iodide (AchI) and 0.4 mM of 5,5′-Dithio-bis (2-nitrobenzoic acid) (DTNB). Reaction time period is 150 seconds at temperature of 25° C. The immobilizing experiments are performed by using electric field intensity of 100 volts and 50 volts for 15 minutes. The Acetylcholinesterase (AchE) is fixed on the support and monitored by a light with wavelength 405 nm. Absorbance at 405 nm measurements indicates that activity of the Acetylcholinesterase (AchE) in the electric field of 100 volts is much greater than in the electric field of 50 volts. Compared with the general vacuum absorption approach, same results also are obtained. Since the invention only takes 15 minutes, this proves that the invention can greatly shorten the immobilization time in which incubation method takes a few hours and vacuum suction approach takes 30 minutes. While the preferred embodiments of the invention have been set forth for the purpose of disclosure, modifications of the disclosed embodiments of the invention as well as other embodiments thereof may occur to those skilled in the art. Accordingly, the appended claims intend to cover all embodiments which do not depart from the spirit and scope of the invention.

1. An electric driven protein immobilizing method comprising steps of:
preparing a solution environment by adding a buffer solution in a container;
preparing a protein/enzyme solution which has an isoelectric point (pI) different from the pH of the solution environment so that the protein/enzyme become to have electric charges in the solution environment;
selecting a support which has electric charges on the surface thereof opposite to the electric charges of the protein/enzyme; and
applying an electric field on the solution environment to accelerate movement of the protein/enzyme in a selected form towards the support so that the protein/enzyme is immobilized on the support by adsorption or bonding.

2. The method of claim 1, wherein the electric field intensity of the external electric field ranges from 80 to 200 volts.

3. The method of claim 1, wherein the electric current density of the external electric field ranges from 38 to 113 mA.

4. The method of claim 1, wherein the buffer solution is selected from Phosphate buffer, Tris buffer or HEPES buffer, or combinations thereof.

5. The method of claim 4, wherein the buffer solution has a pH value ranged from 7 to 11.

6. An electric driven protein immobilizing module for pouring buffer and adding protein/enzyme solution into a module and connect with electrode to form a circuit to form a passage, comprising:
an upper tank having an open upper end and an open lower end, housing a first electrode, and having a bottom formed a plurality of sample wells;
a lower tank being closed and having one end communicating with the upper tank, and housing a separated second electrode, and having apertures under and corresponding to the sample wells;
an electric field generating element connecting respectively to the first electrode and the second electrode;
a silicon rubber pad interposed between the upper tank and the lower tank, the silicon has a plurality of ports aligned with the sample wells and the apertures of the lower tank;
a selected support fixedly located on the silicon rubber pad; and
a buffer solution pouring into a container formed by the upper tank and the lower tank to form a solution environment, the buffer solution being at a level submerged the first electrode in the upper tank.

7. The electric driven protein immobilizing module of claim 6, wherein the electric field generating element is a conductive metal wire.

8. The electric driven protein immobilizing module of claim 6, wherein the electric field generating element is a metal plate.

9. The electric driven protein immobilizing module of claim 6, wherein the selected support is a porous membrane.

10. The electric driven protein immobilizing module of claim 6, wherein the selected support is porous powders.

11. The electric driven protein immobilizing module of claim 6, wherein the selected support is porous granules.
